(19) United States
(12) Patent Application Publication
(10) Pub. No.: US 2006/0100303 A1
(43) Pub. Date: May 11, 2006

(54) Air freshing device

(76) Inventors: David Bedford, East Yorkshire (GB); Paul William Robinson, Lane Hull (GB)

Correspondence Address: NORRIS, MCLAUGHLIN & MARCUS, 875 THIRD AVE, 18TH FLOOR, NEW YORK, NY, 10022, US

(21) Appl. No.: 10/541,887

(22) PCT Filed: Jan. 12, 2004
(86) PCT No.: PCT/GB04/00067

§ 371 Date: Jan. 9, 2006

(30) Foreign Application Priority Data
Jan. 11, 2003 (GB) 0300641.8

(51) Int. Cl. A61L 9/04

(52) U.S. Cl. 523/102

(57) ABSTRACT

Improvements in or relating to devices A device for perfuming, deodorising or sanitising air or enclosed spaces which comprises: a. an anhydrous gel element formed by cross-linking a functionalised liquid polymer with a crosslinking agent in the presence of a non-aqueous perfume, deodorising or sanitising base; and b. separate timing means for proving an end-of-life indication.

The present invention devices useful for the perfuming, deodorising or sanitising of air with an end-of-life indicator.

Devices for perfuming, deodorising or sanitising of air are known. For example, air fresheners are now found in many rooms of houses or offices or in enclosed spaces such as vehicles. Recently devices comprising a high proportion of fragrance in a gel have been marketed under the trade mark “Crystal Air” by Reckitt Benckiser Plc. Such devices are also described in, for example, U.S. Pat. No. 5,780,527, GB-A-2,363,717 and WO 00/24434. Typically the air freshener, deodoriser or sanitising element comprises above 50 wt % active component in a matrix of a crosslinked, functionalised liquid polymer held in a device such as a plastic or grooved glass substrate. As the device is used, the active component is released into the atmosphere. After a certain time, the active component is substantially used up, or is released at an insufficient rate.

For many types of air freshener, there is an effective end-of-life indicator. For example, with an electrically driven device in which a liquid composition is heated and evaporated, it can easily be seen when the liquid reservoir is empty. A perfuming candle clearly needs replacing when it has burnt away. It is difficult for a user of a device comprising a high proportion of an active component in a gelled matrix to determine when it needs replacing since there is no clear end-of-life indicator.

WO 00/24434 states that the end point cue is provided by the shrinkage of the gel material within the recess or recesses, which is perfectly visible. It also states that there is often cracking or tearing of the gel material. However, we have found that the shrinkage or cracking of the gel is not always a reliable indicator of end-of-life. Shrinkage is a continuous process, and it cannot easily be determined when the gel has sufficiently shrunk that the device should be replaced. Furthermore, cracking does not always occur, and when it does, the time of cracking is not necessarily the time that the device ceases to effectively fulfill its function.

The present invention provides a device for perfuming, deodorising or sanitising air or enclosed spaces which comprises:

a. an anhydrous gel element formed by cross-linking a functionalised liquid polymer with a crosslinking agent in the presence of a non-aqueous perfume, deodorising or sanitising base; and

b. separate timing means for proving an end-of-life indication.

The anhydrous gel element is described, for example, in U.S. Pat. No. 5,780,527, GB-A-2,363,717 and WO 00/24434, herein incorporated by reference. By the term “functionalised liquid polymer” we mean a material which is liquid at room temperature and which has a viscosity of not more than 5 Pas at 25° C., preferably from 0.25 to 1.0 Pas. Thus, for example, the functionalised liquid polymer may be selected from maleinised polybutadiene, maleinised polyisoprene or a copolymer of ethylene and maleic anhydride and the crosslinking agent contains at least two complementary functional groups. Examples of functionalised liquid polymers are commercially available from Revertex Ltd and sold under the trade mark LITHENE. Particularly preferred are LITHENE N4-9000 10 MA, LITHENE N4-B-10 MA and LITHENE N4-5000-10 MA. Examples of crosslinking agents are alkylpropyldiamines having an ethoxylated or propoxylated higher aliphatic chain such as those sold under the trade mark DICRODAMET by Croda Chemicals Ltd, ethoxylated or propoxylated primary fatty amines sold under the trade mark CRODAMET and polyoxyalkylenediamines such as those sold under the trade mark JEFFAMINE by Huntsman Corporation.

The non-aqueous perfume, deodorising or sanitising base generally constitutes 50 to 95wt %, preferably 60 to 90wt %, more preferably 70 to 85wt %, of the anhydrous gel element. The gel element may also comprise other components such as dyes or plasticisers.

The timing means provides the user of the device with an indication as to when the device ceases to operate effectively and must be replaced or refilled. Any timing means separate from the anhydrous gel element can be provided. In other words, the anhydrous gel element is not itself used to provide an end-of-life indication.

The timing means desirably provides an end-of-life indication 14 to 60 days from the date that the device is initiated, preferably 28 to 40 days. The duration will, of course, depend on various factors such as the composition of the anhydrous gel element, the physical form of the device and the location of the device when it is used. Desirably the time to the end-of-life indication is set by the manufacturer of the device, but it is also possible for a user to set the duration if suitable adjustment means are provided.

The timing means is preferably actuated when the device itself is initially actuated by a single operation. However, it is possible for separate actuation means to be provided for the device and for the timing means.

Any timing means can be provided so long as it shows the desired end-of-life indication. For example, a visible or audio signal can be provided.

In a first embodiment, a further gel element can be provided. The further gel element has a different composition from the anhydrous gel element, in that it is specifically designed to shrink. By anchoring the further gel element at one end, the shrinkage of the element to the anchor point can be seen. The end-of-life indication is provided when a certain degree of shrinkage is observed, for example by the gel shrinking past an indicator such as a line embossed on the device. Desirably the further gel element is not perfumed, but it is preferably coloured to provide an attractive and highly visible appearance. It may take the form of a simple line, or a more complex shape could be used, for example a circle or circular element around the anhydrous gel element to provide a timing means reminiscent of a stopwatch face. Such an element comprises a composition able to shrink over time, for example of gelatin or carrageenan gum.

In a second embodiment, a composition which compacts, changes state or dissolves can be provided. For example, a composition can be in the form of discrete particles, preferably spherical particles. The particles can be held in a reservoir, preferably a substantially vertical reservoir, and slowly decay by evaporation of water and eventual coalescence at the bottom of the reservoir. The end-of-life indication can be when discrete particles are no longer visible, or when the level of particles reaches an indicated level.

Any composition fulfilling the above requirement can be used, but of particular benefit is a copolymer of a substituted or unsubstituted maleic anhydride with a substituted or unsubstituted alkene. Examples of substituted or unsubstituted maleic anhydrides are those of formula:

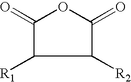

wherein R₁ and R₂, which may be identical or different, are each hydrogen or alkyl, especially C₁-C₆ alkyl. Preferably R₁ and R₂ are both hydrogen.

Examples of suitable alkenes are straight branched C₂-C₁₂ alkenes especially C₂-C₆ alkenes, and in particular C₄ alkenes such as isobutylene.

The copolymer is preferred in the form of salt, especially an alkali metal salt such as a sodium salt.

A particularly preferred copolymer is isobutylene-maleic anhydride sodium copolymer obtainable from Kuraray, Osaka, Japan.

In order to obtain particles containing water the copolymer particles are simply added to water and allowed to swell. Ordinarily the swelling is allowed to proceed to completion, but controlled swelling can be used by adding a restricted amount of water or by removing the particles before swelling is completed. In this way the end-of-life timing can be controlled, It can also be controlled by an appropriate choice of copolymer, or by incorporating a water-permeable membrane between the timing means and the atmosphere to retard the loss of water from the particles.

In a third embodiment an audio signal can be provided, for example by an electrical timing circuit, preferably powered by a battery, connected to a loudspeaker. An example of this embodiment is the same as the first or second embodiments above except that the composition is conducting, by inclusion of conducting particles such as carbon black, graphite or a metal. Once the composition passes a particular point, for example by shrinkage of a gel or dissolution of particles, a circuit could be broken. For example, two electrodes can be provided, and once the circuit between them is broken, a signal can be generated.

Examples of two devices constructed in accordance with the present invention will now be described with reference to the accompanying drawings, in which:

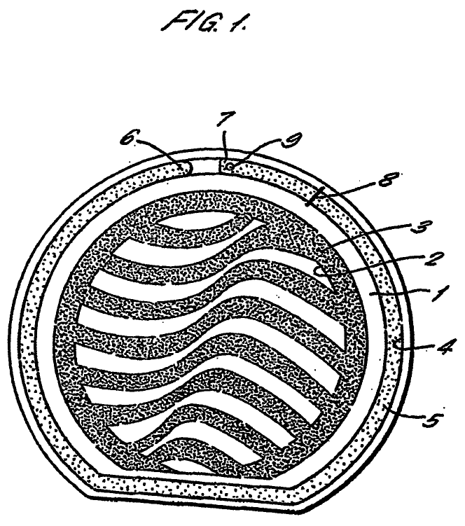

FIG. 1 is a plan view of an example of the first embodiment of a device of the present invention; and

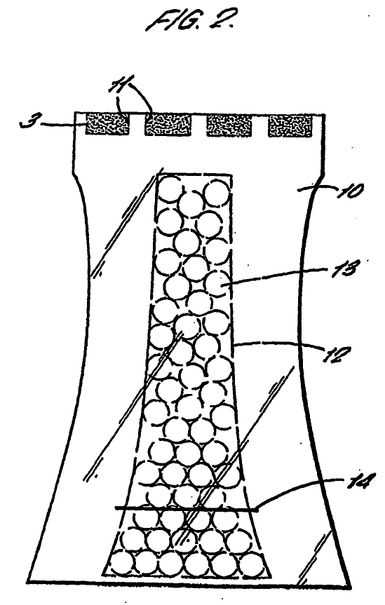

FIG. 2 is a plan view an example of a second embodiment of a device of the present invention.

FIG. 1 illustrates an adapted version of the Crystal Air (RTM) product sold by Reckitt Benckiser Plc.

This device comprises a glass body 1, one face of which is provided with a pattern of grooves 2 in which a fragranced gel 3 is deposited. The grooves are open to the ambient air such that the gel fragrances the air.

This device has now been adapted to have a further groove 4 separate from and running around the periphery of the pattern of grooves 2. This further groove 4 is filled with a second gel 5 which extends along most of the length of the groove 4, but does not extend around the entire groove. The gel 5 is anchored in position at a first end by anchoring means such as a spike 9 and has an opposing free end 7. A marker 8 is provided adjacent to the groove 4 at a certain distance behind the free end 7.

In use when the device is activated, for example by removing a protective foil from the back of the device which covers both the pattern of grooves 2 and the further groove 4, or by removing the entire device from an air-tight container, the second gel 5 begins to shrink, such that the free end moves towards the marker 8. Eventually, the free end 7 will reach the marker 8 to provide an end-of-life indication.

In the second example shown in FIG. 2, the same fragranced gel 3 is used as in the first example, but this time in a reservoir on top of the upper end of a housing 10. The gel is held in a plurality of grooves 11 to allow fragancing in a similar way as in the first example

The housing has a vertically extending chamber 12 which is externally visible, either by the housing itself being transparent or through a transparent window.

The chamber 12 is filled with a stack of spherical balls 13. These are made of a material which is designed to decay when exposed to ambient air. For example, they may be impregnated with water which evaporates over time such that the resulting structure is incapable of supporting its own mass and collapses, or the balls shrink as water is released.

Thus, a given period of time after the device is activated, for example by removing the device from an air tight container, the balls 13 will collapse into a small space at the bottom of the chamber 12. A marker 14 is preferably provided towards the bottom of the chamber 12 such that, the end-of-life is indicated by the balls collapsing below the marker 14. Although FIG. 2 shows the chamber being transparent beneath the marker 14, this need not necessarily be the case, and the end-of-life may be indicated by the balls 13 disappearing from view.

1. A device for perfuming, deodorising or sanitising air or enclosed spaces which comprises:
a. an anhydrous gel element formed by cross-linking a functionalised liquid polymer with a crosslinking agent in the presence of a non-aqueous perfume, deodorising or sanitising base; and
b. separate timing means for proving an end-of-life indication.

2. A device according to claim 1 wherein the functionalised liquid polymer is selected from maleinised polybutadiene, maleinised polyisoprene or a copolymer of ethylene and maleic anhydride and the crosslinking agent contains at least two complementary functional groups.

3. A device according to claim 1 wherein the non-aqueous perfume, deodorising or sanitising base constitutes from 60 to 95 wt % of the gel element.

4. A device according to claim 1 wherein the timing means comprises an additional element which visibly shrinks over the intended lifetime of the device.

5. A device according to claim 4 wherein the additional element comprises a gel which shrinks at a greater rate than said anhydrous gel element.

6. A device according to claim 4 wherein the additional element comprises particles which shrink, compact or dissolve.

7. A device according to claim 6 wherein the particles comprise a maleic anhydride-isobutylene copolymer.

8. A device according to claim 1 wherein the timing means provides an end-of-life indication after from 28 to 40 days use of the device.

9. A device according to claim 1 which comprises actuating means which, when actuated, activates the device to release the perfuming, deodorising or sanitising substance and activates the timing means.

10. A device according to claim 9 wherein the actuating means comprises an impermeable foil which covers both the perfuming, deodorising or sanitising substance and the timing means.

11. A device according to claim 2 wherein the non-aqueous perfume, deodorising or sanitising base constitutes from 60 to 95 wt % of the gel element.

12. A device according to claim 1 wherein the separate timing means for proving an end-of-life indication is a visible signal means.

13. A device according to claim 1 wherein the separate timing means for proving an end-of-life indication is an audio signal means.

14. A device according to claim 1 wherein the anhydrous gel element comprises conducting particles.

15. A device according to claim 14 wherein the conducting particles are selected from carbon black, graphite and metals.
